(19) United States
(12) Patent Application Publication
(10) Pub. No.: US 2004/0210245 A1
(43) Pub. Date: Oct. 21, 2004

(54) Bendable needle with removable stylet

(76) Inventors: John Erickson, Plano, TX (US); Terry Daglow, Allen, TX (US)

Correspondence Address: Koestner Bertani LLP, P.O. Box 26780, Austin, TX, 75755, US

(21) Appl. No.: 10/627,233

(22) Filed: Jul. 25, 2003

Related U.S. Application Data
(60) Provisional application No. 60/400,366, filed on Aug. 1, 2002.
(60) Provisional application No. 60/398,704, filed on Jul. 26, 2002.
(60) Provisional application No. 60/398,749, filed on Jul. 26, 2002.
(60) Provisional application No. 60/398,740, filed on Jul. 26, 2002.

(51) Int. Cl. A61B 17/32

(52) U.S. Cl. 606/167

(57) ABSTRACT

The invention is directed to a bendable needle with a removable stylet. Needles are typically used in the practice of inserting electrodes and other small instruments into patients. Stylets fit flush with the cutting end of the needle to prevent tissue from entering the needle. This invention is directed to a needle that may be bent or shaped to better suit a specific application. Typical stylets are irremovable from a bent needle. However, the present invention incorporates a stylet with a coil, flexible tube, and/or flexible rod attached to a tip. The tip fits flush with the cutting end of the needle and the coil, tube, or rod extends through the bore of the needle. As such, the stylet is removable from the needle once bent.

### RELATED APPLICATIONS

This application claims the benefit of U.S. Provisional Patent Application No. 60/400,366 entitled, “Bendable Needle with Removable Stylet” filed Aug. 1, 2002. Additionally, this application incorporates by reference the prior U.S. provisional application Nos. 60/398,704 entitled, “Method and System for Energy Conservation in Implantable Stimulation Devices” filed Jul. 26, 2002; 60/398,749 entitled, “Method and Apparatus for Providing Complex Tissue Stimulation Patterns” filed Jul. 26, 2002; and 60/398,740 entitled, “High Frequency Pulse Generator for an Implantable Neurostimulator” filed Jul. 26, 2002.

### TECHNICAL FIELD OF THE INVENTION

This invention relates in general to a needle used for insertion of devices into corporal locations. More specifically, the invention relates to a needle that may be bent or shaped and a removable stylet.

### BACKGROUND OF THE INVENTION

Needles may typically be used to provide a path for the insertion of certain devices into patients. Among other uses, needles may be used in the placement of electrodes in the epidural space about the spinal cord, laproscopic surgery, or the insertion of catheters. Needles used in these procedures are often large enough that a stylet is necessary during needle insertion

In many instances, no direct path exists to the corporal location at which the device is to be placed. Bones, organs, or sensitive tissues may block the path to the desired location of the device. As such, the desired location may be approached circuitously. For this reason, needles may be bent or shaped to avoid harming the patient or the needle.

When such a needle is bent, often by the attending physician at the time of surgery, a problem arises in removing the stylet. A tip of the stylet fits flush with the tip of the needle. As the needle is inserted, the stylet prevents encroachment of tissue into the needle. After insertion, the stylet may be removed from the needle, leaving an unobstructed path through which the device, instrument, or second needle may pass.

However, in these typical needles and stylets, removal of the stylet is difficult from a bent or shaped needle. Typically, the stylet either is irremovable or provides significant resistance to removal. Typical solutions to this problem cause the tip of the stylet to move during needle insertion causing undesirable incursion of tissue into the needle. This incursion may block or obstruct the path or bore through the needle.

As such, many insertion needles and methods suffer from deficiencies in adaptability to procedures and anatomy. Many other problems and disadvantages of the prior art will become apparent to one skilled in the art after comparing such prior art with the present invention as described herein.

### SUMMARY OF THE INVENTION

Aspects of the invention are found in a needle for insertion of devices about a corporal location. The needle may have a hollow section leading to a sharp cutting end. The other end of the needle may be attached to a hollow fitting. Together, the sharp cutting end, hollow section, and hollow fitting may form a continuous bore through which devices may be inserted. A stylet may be inserted through the bore of the needle. The stylet may have a tip attached to a coil, flexible rod, or flexible tube. The tip may fit flush with the sharp cutting end of the needle and the coil, flexible rod, or flexible tube may extend into the bore from the tip. In this manner, the stylet may prevent encroachment of tissue into the bore of the needle. Subsequent to insertion, the stylet may be removed from the needle thereby providing a path through the bore for insertion of a device.

Another aspect of the invention may be found in the stylet. The stylet may have a tip attached to a coil, flexible rod, or flexible tube. The tip may be manufactured to fit flush with the sharp end of a needle. A fitting may be attached to the coil, flexible rod, or flexible tube opposite the tip. The fitting may selectively couple to a hollow fitting of the needle. In this manner, the fitting may hold the stylet in place while the needle is inserted into a patient.

Another aspect of the invention may be found in a rod locate in and concentric with the coil or flexible tube. The rod may be attached to the tip and the fitting.

A further aspects of the invention may be found in a method for inserting a device about a corporal location. The method may involve bending the needle to a desired shape or flexing the needle during insertion. Once the needle is inserted, the stylet may be removed and the device inserted. Subsequently, the needle may be removed. The device may or may not remain about the corporal location.

As such, a needle, stylet, and method used to insert a device about a corporal location is described. Other aspects, advantages and novel features of the present invention will become apparent from the detailed description of the invention when considered in conjunction with the accompanying drawings.

### BRIEF DESCRIPTION OF THE DRAWINGS

For a more complete understanding of the present invention and advantages thereof, reference is now made to the following description taken in conjunction with the accompanying drawings in which like reference numbers indicate like features and wherein:

FIGS. 1A and 1B are schematic diagrams of a needle according to the invention;

FIG. 2 is a schematic diagram of a needle and stylet according to the invention;

FIGS. 3A and 3B are schematic diagrams of an exemplary embodiment of the stylet as seen in FIGS. 1 and 2;

FIGS. 4A, 4B, 4C, 4D, and 4E are schematic diagrams of an exemplary embodiment of the stylet seen in FIG. 3;

FIG. 5A is schematic diagram of an exemplary embodiment of the stylet and needle as seen in FIG. 1;

FIG. 5B is schematic diagram of another exemplary embodiment of the stylet and needle as seen in FIG. 1;

FIG. 5C is schematic diagram of a further exemplary embodiment of the stylet and needle as seen in FIG. 1;

FIG. 6 is a schematic diagram of an exemplary embodiment of the needle as seen in FIG. 1;

FIG. 7A is a schematic diagram of an exemplary embodiment of the stylet as seen in FIG. 3;

FIG. 7B is a schematic diagram of an exemplary embodiment of the stylet as seen in FIG. 3;

FIG. 8 is a schematic diagram of an exemplary embodiment of the stylet as seen in FIG. 3;

FIG. 9 is a block flow diagram of an exemplary method for using the needle as seen in FIGS. 1 and 2; and

FIG. 10 is a schematic diagram of an exemplary use of the method as seen in FIG. 9.

Corresponding reference numerals indicate corresponding parts throughout the several views of the drawings.

### DETAILED DESCRIPTION OF THE PREFERRED EMBODIMENT

Various procedures require the insertion of devices into patients. These devices are often inserted through the use of needles or needle-like instruments. Some techniques require insertion of a second needle through a first needle. Large diameter needles with stylets are used for these procedures. During needle insertion, the stylet acts to prevent tissue from entering the hollow portion of the needle and prevent blocking the path subsequently used for insertion of the device. Once the needle is in place, the stylet is typically removed, providing an unobstructed path for the insertion of the device.

Many times, bones, organs, or other sensitive tissues prevent direct access to the desired insertion location. In these cases, access often may be achieved by placing an arc along some portion of the needle to approach the insertion position from a circuitous path. At other times, an arced or bent needle may aid in directing the insertion in a particular direction.

For example, a bent needle may aid in the placement of electrodes in the spinal foramen. Typically, accessing the spinal foreman is difficult because it is protected by vertebrae. In addition, it may be of benefit to direct the electrode in a superior, inferior, or transverse direction in or about the spinal foreman. Additionally, a bent needle may be used in the retrograde placement of an electrode lead in the spinal foramen or directing an electrode lead in an inferior direction. These leads may include SCS leads or neuromodulation leads.

However, various other applications may be envisaged for a bent or bendable needle with a removable stylet. Furthermore, various embodiments of the invention may be envisaged given the description below.

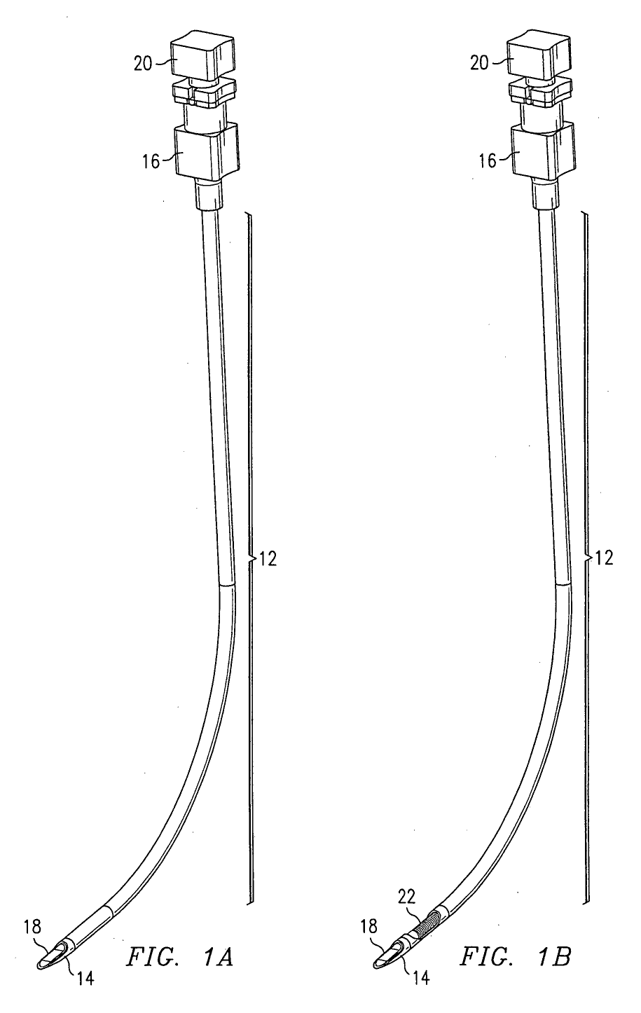

FIGS. 1A and 1B depict exemplary embodiments of a bendable needle with a stylet. The needles may have a longer hollow section 12 with a sharp cutting end 14. A hollow fitting 16 may be attached to the hollow section 12 opposite the sharp cutting end. Together, the sharp cutting end 14, hollow section 12, and hollow fitting 16 may form a continuous bore through the needle.

In the needle may be a stylet. The stylet may have a tip 18 connected via a bendable or flexible material or structure 22 to a fitting 20. The fitting 20 may connect with the hollow fitting 16, so that the stylet may be held in place while the needle is inserted. The stylet may be subsequently removed to allow for subsequent use of the hollow bore of the needle to insert medical devices. The tip 18 of the stylet may fit flush with the sharp cutting end 14 of the needle.

The needle may be formed of various metals, alloys, composites, or plastics, among others. For example, the needle may be formed of surgical steel, biocompatible plastic, or combinations, among others. The stylet may also be formed of various metals, alloys, composites, plastics, or combinations, among others. For example, the stylet may be formed of surgical steel or biocompatible plastic among others. In addition, the needle and stylet may have cross-sections with various shapes. In one exemplary embodiment, the needle may be formed of surgical steel and the stylet may have a surgical steel tip and coil. In this exemplary embodiment, the needle and stylet may have a cylindrical shape and circular cross section.

However, the needle and stylet may take various forms. These elements may or may not be included and may be together, separate, or in various configuration. Further, the needle may bend to form various shapes and curves and may have multiple bends.

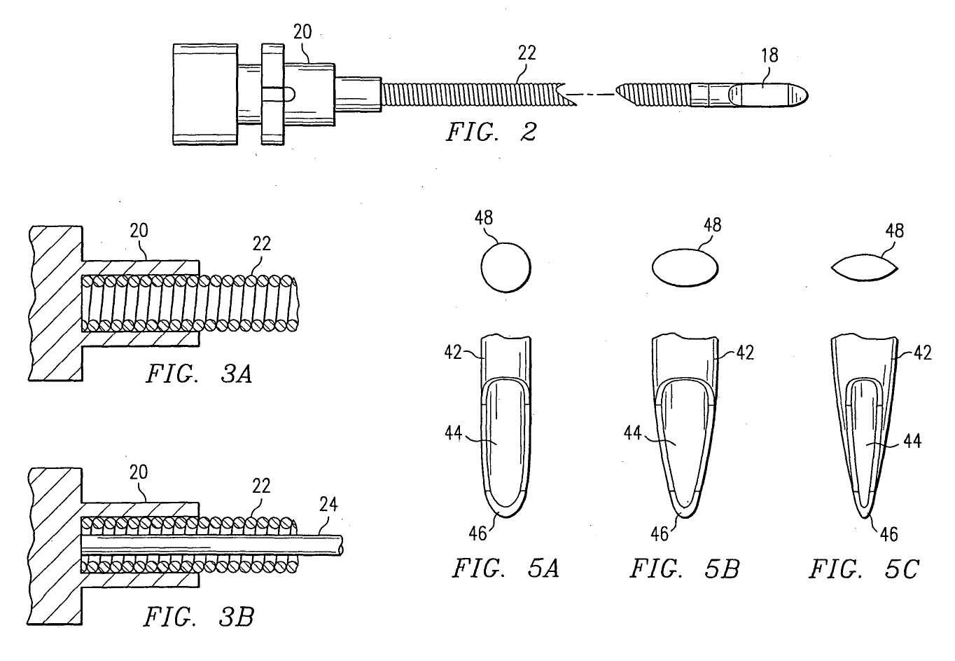

FIG. 2 depicts an exemplary embodiment of a stylet. The stylet has a tip 18, a coil 22 and a fitting 20. The tip 18 is coupled to the coil 22. The coil 22 is coupled to the fitting 20. The coil shape of the stylet body allows the stylet to bend or flex regardless of the arc of the needle. Many times the physician will need to bend the needle during the course of the surgery to account for the patient's body. The physician may use a small apparatus to bend the needle. However, the physician may also bend the needle by hand.

The stylet may function to ensure that the cross-section of the hollow portion of the needle does not collapse at the arc during the bending of the needle. The bent portion must allow for the rigid tip to pass through when the stylet is removed.

Therefore, the coiled shape of the stylet body may perform two functions. First, it may provide a structure in its cross-section, thereby providing support to the inner diameter of the hollow needle. In this manner, it may aide in maintaining the inner diameter of the inner portion of the needle, so that the rigid tip may be removed subsequent to arcing the needle. Secondly, once the needle is arced, the coiled shape of the stylet may provide the stylet with a flexible shape for removal in a longitudinal direction. In this manner, the coiled structure of the stylet may facilitate the removal of the stylet with minimum force.

FIG. 3A shows an exemplary embodiment in which the coil 22 has a hollow center and is coupled to the fitting 20. Alternately, a rod 24 may be connected to the fitting 20 through the hollow center of the coil 22 as seen in FIG. 3B. However, various embodiments may be envisaged. The rod 24 may or may not be coupled to the fitting 20 or the tip. With the rod 24, the coil 22 may or may not be coupled to the fitting 20 or the tip 18. Further other means may be used to extend between the tip and fitting such as rods, tubes, and other coil configurations and combinations.

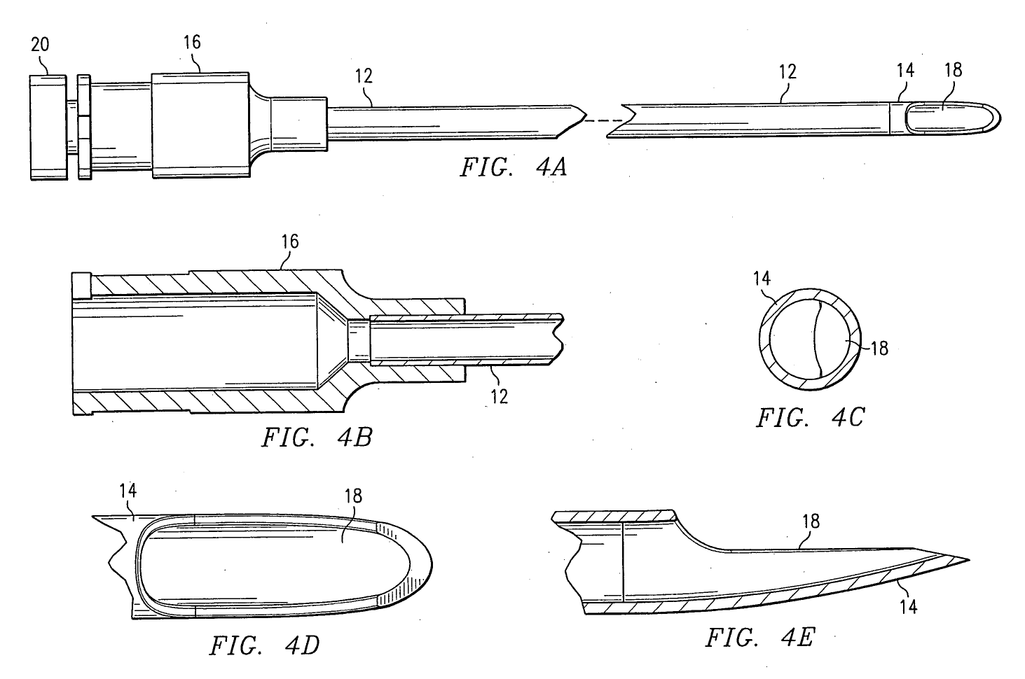

In FIGS. 4A, 4B, 4C, 4D and 4E, an embodiment of the stylet and needle are further demonstrated. FIG. 4A depicts a cylindrical needle with the hollow section 12 between a sharp end 14 and the hollow fitting 16. Here too, the tip 18 of the stylet is shown to fit flush with the sharp cutting end 14. A fitting 20 may be attached to a coil or rod (not shown) that are coupled to the tip and extend inside the needle. FIG. 4B depicts the hollow fitting connected to the hollow section 12 forming a continuous bore through which the stylet may pass.

FIGS. 4C, 4D, and 4E depict an exemplary embodiment of the tip 18 of the stylet and the sharp cutting end 14 of the needle. FIG. 4C depicts a view through the hollow section 12 of the needle toward the stylet. This pictorial depicts the tip 18 of the stylet fitting against the wall of the cutting end 14. Similarly, FIGS. 4D and 4E depict the tip 18 fitting flush with the sharp cutting end 14.

Further, other shapes of the tip, stylet, and needle may be envisaged. For example, FIG. 5A depicts a cylindrical shaped needle 42 and stylet. The needle has a stylet tip 44 fitted to the sharp end 46. The cross section 48 is represented as a circle. Alternately, a needle with an oval-like cross-section 48 may be envisaged as seen in FIG. 5B. In another embodiment, the sharp end 46 may be tapered as seen in FIG. 5C. In various embodiments, the cross-section of the needle may take forms in which the distance of the wall of the needle from a center axis is not uniform. Such forms include those with cross-sectional shapes such as ovals, triangles, squares, rectangles, banana-shapes, cardioid, hexagon, diamond, and polyhedron, among others. The shape of the tip may also take various forms conforming to a machined or sharpened edge of such shapes.

Incursion of tissue into the needle is a particular problem of larger diameter needles. For example, incursion may occur in a Touhy-like needle during insertion if a stylet is absent. However, with a stylet, encroachment of tissue can be prevent. Similarly, other large diameter needles and other needles of various diameters may have a stylet.

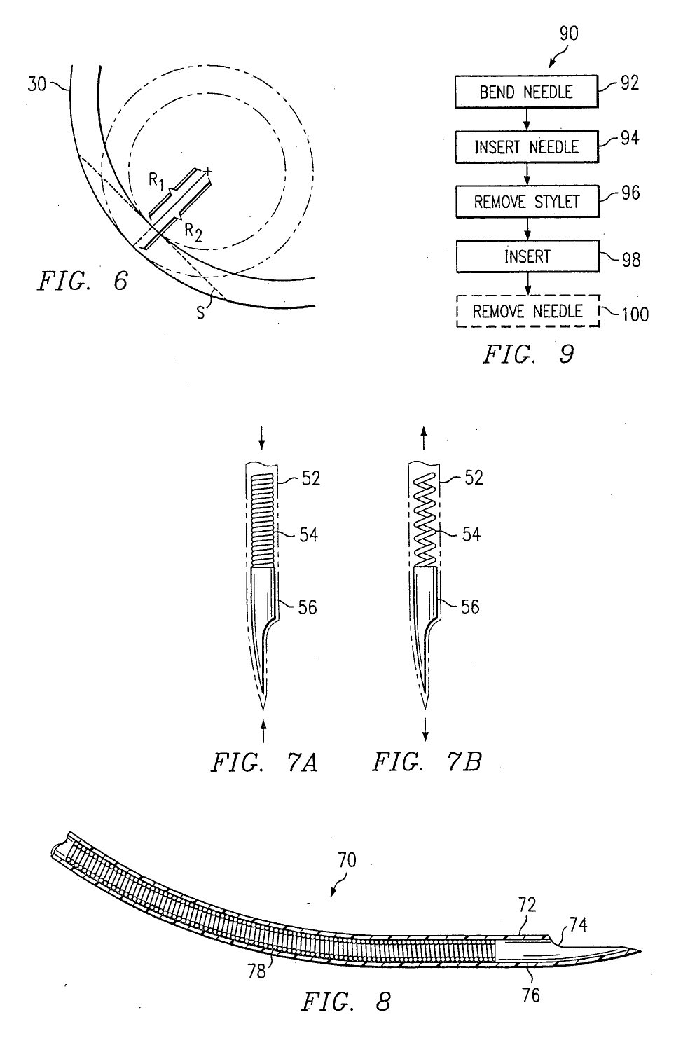

After insertion, the stylet may be removed. However, in some cases, the needle may be bent to facilitate insertion into a particular corporal location. In these cases, the stylet must be removable given the curvature of the needle. FIG. 6 depicts a cross-sectional view of an exemplary embodiment of a bent needle 30. At the bend, the needle has a radius of curvature. The radius R1 at the wall closest to the center point is smaller than that furthest R2. The difference in radii results in a secant S traversing the outside curve and running tangential to the wall of the inside curve. A stylet with a body that has rigid structural elements larger than the secant S will resist extraction through the needle. As such, such stylets will either require great force to remove them from the needle or will be irremovable from a needle bent to have a small radius of curvature.

However, stylets with substantially flexible elements may be removed from needles with smaller radii of curvature. FIGS. 7A and 7B depict one exemplary embodiment. In this exemplary embodiment, a coil 54 may be connected to a tip 56 and inserted into a needle 52. The coil may, for example, be a tightly wound coil so that in stasis, each successive loop touches the previous one. In compression, as seen in FIG. 7A, the coil 54 holds the tip 56 in place. This compression may, for example, occur when a fitting attached to the end of the coil 54 opposite the tip 56 is coupled to a fitting or the needle 52 and the needle 52 is being inserted into a corporal tissue.

However, when the stylet is pulled from the needle 52 or a tension is placed on the stylet, the coil 54 may dynamically extend as seen in FIG. 7B. Alternately, the coil rings may shift to accommodate the curvature of the needle 52. This accommodation may be seen in FIG. 8.

FIG. 8 depicts a bendable needle apparatus 70. A stylet having a coil 78 and a tip 76 is situated in a needle 72. The tip 76 of the stylet fits flush with the sharp end 74 of the needle 72. In FIG. 8, the coil 78 is shown to adapt to the shape of the bent needle 72. This adaptation of the coil 78 and/or a dynamic extension process enable the coil 78 to be removed from the bent needle 72.

However, various embodiments may be envisaged with varying mechanisms for extracting a stylet from a bent needle. Coils, flexible tubes, and flexible rods may be used separately or in combinations. For example, a plastic tube or rod may or may not replace the coil.

The extraction of the stylet from the bent needle may enable various surgical methods or method for inserting or implanting devices. FIG. 9 depicts an exemplary method for inserting a device about a corporal location. In the method 90, the needle and/or stylet may be bent as seen in a block 92. Bones, organs, or other sensitive regions may prevent direct or linear access to a location within the body. Additionally, the device may require a specific direction of insertion as dictated by the direction at the point of the need.

The needle with the stylet may then be inserted as seen in a block 94. In this manner, a physician may direct the sharp cutting end of the needle to a desired corporal location. The stylet may then be removed as indicated by block 96. As a result, a path through the bore of the needle for insertion of the device is maintained and tissue is prevented from encroaching the inside of the needle.

The device may then be inserted through the bore of the needle as seen in a block 96. As such, the needle acts as a guide for positioning or inserting the device in the desired location. Subsequently, the needle may be removed, leaving the device in place or after removing the device as well.

One exemplary use of this method is the insertion of electrodes in the epidural space about the spinal column. Electrodes may be used to treat various conditions such as chronic pain and symptoms of motor dysfunction. Typically, the electrodes are placed near specific nervous tissue in or about the spinal cord. The electrodes impart various electrical pulses and signals to the nervous tissue. In doing so, the electrodes may initiate and maintain paresthetics or block nerve signals, among others.

However, the electrodes are typically inserted as leads in the spinal column. The spinal column is protected by vertebrae. Anterior, the vertebrae have extending processes and lamina. Posterior, the vertebrae have a bone body. As such, access to the spinal cord and the spinal foramen is limited by these solid structures. However, the spinal foramen may be access through peripheral foramen or between lamina of adjacent vertebrae and the lead directed in the superior or inferior directions with a bent needle.

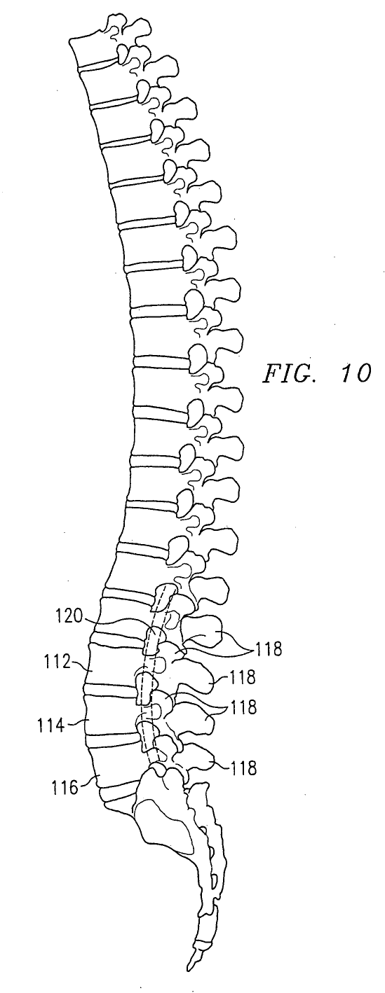

FIG. 10 depicts the spine and spinal column. In one exemplary embodiment electrode leads may be inserted in the spinal foreman 120 about the 3rd, 4th or 5th lumbar vertebrae. However, posterior access is limited by the bone bodies (112, 114, 116, respectively) of these vertebrae. In addition, anterior access is limited by various processes and laminae 118. Furthermore, it may be desirable to direct the leads in a superior or inferior direction inside the spinal foreman. In this case, a bent needle with removable stylet may be useful.

For example, a needle that may be bent to hold a specific shape may be useful in retrograde insertion of neuromodulation leads and direction of leads in an inferior direction. However, the electrode may be inserted in various directions between various vertebrae. Similarly, various devices may be inserted into various locations within a body.

As such, a bendable needle with removable stylet and method for using such is described. In view of the above detailed description of the present invention and associated drawings, other modifications and variations will now become apparent to those skilled in the art. It should also be apparent that such other modifications and variations may be effected without departing from the spirit and scope of the present invention as set forth in the claims which follow.

What is claimed is:

1. A bendable needle system, comprising:
a bendable tubular needle having at least one a sharp cutting end; and
a removable stylet inserted in a hollow interior of the bendable tubular needle, wherein the removable stylet further comprises:
a flexible stylet body that allows the stylet to bend within the bendable tubular needle; and
a tip that aligns flush to the sharp cutting end of the bendable tubular needle.

2. The bendable needle system of claim 1, further comprising a fitting coupled to a non-cutting end of the hollow interior of the needle.

3. The bendable needle system of claim 2, wherein the fitting comprises a handle, and wherein an object may be inserted into the hollow interior of the bendable tubular needle via a passage within the fitting.

4. The bendable needle system of claim 1, wherein the flexible stylet body further comprises a coil.

5. The bendable needle system of claim 1, wherein the flexible stylet body further comprises a flexible rod.

6. The bendable needle system of claim 1, wherein the flexible stylet body further comprises a flexible tube.

7. The bendable needle system of claim 1, wherein a cross-section of the hollow interior comprises a shape in which all points along the shape's perimeter are not equidistant from a longitudinal axis of the bendable tubular needle.

8. A removable stylet, comprising:
a flexible stylet body that allows the removable stylet to bend within a bendable tubular needle having at least one a sharp cutting end, and wherein the removable stylet is inserted within a hollow interior of the bendable tubular needle; and
a tip that aligns flush to the sharp cutting end of the bendable tubular needle.

9. The removable stylet of claim 8, further comprising a fitting coupled to a non-cutting end of the hollow interior of the needle.

10. The removable stylet of claim 9, wherein the fitting comprises a handle, and wherein an object may be inserted into the hollow interior of the bendable tubular needle via a passage within the fitting.

11. The removable stylet of claim 8, wherein the flexible stylet body further comprises a coil.

12. The removable stylet of claim 8, wherein the flexible stylet body further comprises a flexible rod.

13. The removable stylet of claim 8, wherein the flexible stylet body further comprises a flexible tube.

14. The removable stylet of claim 8, wherein a cross-section of the hollow interior comprises a shape in which all points along the shape's perimeter are not equidistant from a longitudinal axis of the bendable tubular needle.

15. A method for inserting a lead in vivo, comprising the steps of:
inserting a bendable needle into living tissue, wherein a removable stylet is contained within a interior cavity within the bendable needle;
guiding the bendable needle to a corporal location, wherein the bendable needle follows a non-straight path.
withdrawing the removable stylet from the bendable needle through a non-cutting end of the bendable needle; and
delivering the lead to the corporal location through the interior cavity of the bendable needle.

16. The method of claim 15, wherein the corporal location is an epidural space about a spinal cord.

17. The method of claim 15, wherein the lead delivers an electric pulse to living tissue(s) through electrodes electrically coupled to a pulse generator via the lead.

18. An electrical pulse stimulation system comprising:
a stimulation device, wherein the stimulation device further comprises:
a microprocessor;
a switching circuit communicatively coupled to the microprocessor;
a pulse generator that generates a repeating pattern of pulses, wherein the pulse generator is communicatively coupled to the microprocessor provided to an output port of the stimulation device;
a lead couple electrically coupled to an output of the stimulation device, wherein the lead is implanted to a corporal location with a bendable needle system; and
at least one electrode within the lead, wherein the at least one electrode delivers an electrical pulse generated by the stimulation device to living tissue at the corporal location and proximate to the at least one electrode.

19. The electrical pulse stimulation system of claim 18, wherein the stimulation device is implantable within a living organism.

20. The electrical pulse stimulation system of claim 18, wherein the stimulation device is a neurostimulator.

21. The electrical pulse stimulation system of claim 18, wherein the bendable needle system further comprises:
a bendable tubular needle having at least one a sharp cutting end; and
a removable stylet inserted in a hollow interior of the bendable tubular needle, wherein the removable stylet further comprises:
a flexible stylet body that allows the stylet to bend within the bendable tubular needle; and
a tip that aligns flush to the sharp cutting end of the bendable tubular needle.
